(19) United States
(12) Patent Application Publication
(10) Pub. No.: US 2022/0280754 A1
(43) Pub. Date: Sep. 8, 2022

(54) SLEEVE FOR AN ENDOSCOPE AND/OR CATHETER INCLUDING A HELICAL DESIGN

(71) Applicant: Covidien LP, Mansfield, MA (US)

(72) Inventors: Paul M. Galluzzo, Godmanchester (GB); Rita Stella, Sandy (GB); Neil Pollock, Wimpole (GB)

(21) Appl. No.: 17/665,447

(22) Filed: Feb. 4, 2022

Related U.S. Application Data
(60) Provisional application No. 63/156,896, filed on Mar. 4, 2021.

(51) Int. Cl. A61M 25/01; A61M 25/00; A61B 1/00

(52) U.S. Cl. CPC A61M 25/0105; A61M 25/0017; A61B 1/0008

(57) ABSTRACT

The present disclosure describes a sleeve having a helical design on at least a distal end portion thereof. The sleeve configured to be used with an endoscope and/or catheter.

### CROSS-REFERENCE TO RELATED APPLICATIONS

This application claims benefit of and priority to U.S. Provisional Patent Application Nos. 63/156,896 filed Mar. 4, 2021, the disclosure of which is hereby incorporated by reference in its entirety.

### BACKGROUND

### Technical Field

The present disclosure is generally related to a sleeve for an endoscope or catheter, and more particularly, a helical sleeve for an endoscope or catheter assembly.

### Description of Related Art

A wide variety of endoscopes and catheters, as well as sleeves designed to be used with such devices, have been developed. Of these known devices, each has certain advantages and disadvantages. However, there is an ongoing need to provide alternative endoscopes, catheters, and/or sleeves. For example, some known endoscopes and/or catheters may have difficulty properly aligning a surgical instrument to a targeted tissue or lesion within or beyond a given bodily lumen. More particularly, some known endoscopes and/or catheters may be unable to properly articulate inside a given tissue lumen or device channel thereby preventing proper alignment of the endoscope or catheter, and ultimately the surgical instrument delivered therethrough. Thus, there exists a need to provide a sleeve which can be easily combined with an endoscope or catheter to more efficiently align the endoscope and/or catheter, and ultimately a surgical instrument, with a target tissue or lesion.

### SUMMARY

The present disclosure describes a sleeve configured to be used with either an endoscope or a catheter. The sleeve is designed to aid in properly aligning a surgical instrument with a target tissue via an endoscope or catheter. The endoscope as described herein may be a bronchoscope.

In some embodiments, a sleeve for an endoscope is described including a sleeve body having a proximal end portion and a distal end portion, at least the distal end portion including a helical sidewall. The helical sidewall defines a plurality of helical bands extending longitudinally to define a longitudinal sleeve channel therein. The longitudinal sleeve channel is configured to receive a catheter therein.

In some embodiments, a sleeve for a catheter assembly is also described. The sleeve includes a sleeve body having a proximal end portion and a distal end portion, at least the distal end portion including a helical sidewall defining a plurality of helical bands extending longitudinally to define a longitudinal sleeve channel therein. The longitudinal sleeve channel is configured to receive a surgical instrument therein.

The sleeve also includes a helical slot following the helical sidewall and separating each of the plurality of helical bands from each other. In some instances, the helical slot may include a plurality of helical gaps, each helical gap separating neighboring helical bands of the plurality of helical bands. In some instances, the plurality of helical bands are separated by the plurality of helical gaps having a common length.

The plurality of helical bands may include one or more proximal helical bands and a distal-most helical band. In some instances, the distal-most helical band defines a length greater than each of the one or more proximal helical bands.

In some instances, a length of each of the one or more proximal helical bands is the same. In some other instances, a length of each of the one or more proximal helical bands increases as the proximal helical bands move distally along the sleeve. In still other instances, a length of each of the one or more proximal helical bands decreases as the proximal helical bands move distally along the sleeve.

In some embodiments, the plurality of helical gaps includes one or more proximal helical gaps and a distal helical gap. The one or more proximal helical gaps are positioned between neighboring proximal helical band. The distal helical gap is positioned between the distal-most proximal helical band of the one or more proximal helical bands and the distal-most helical band.

In some instances, a proximal gap length of each of the proximal helical gaps is the same. In some other instances, a proximal gap length of each of the proximal helical gaps increases as the proximal helical gaps move distally along the sleeve. In still other instances, a proximal gap length of each of the proximal helical gaps decreases as the proximal helical gaps move distally along the sleeve. In some instances, a distal gap length of the distal helical gap is greater than a proximal gap length of the proximal helical gaps.

In some embodiments, the sleeve includes at least one US transducer. In some instances, the distal-most helical band may further include one or more US transducers.

Kits including the sleeves described herein are also provided.

### BRIEF DESCRIPTION OF THE DRAWINGS

Various aspects and features of the present disclosure are described herein below with reference to the drawings, wherein:

FIG. 1A depicts a side view of a sleeve positioned on an endoscope or a catheter as described in at least one embodiment herein;

FIG. 1B depicts a top view of a helical band of the sleeve of FIG. 1A, as described in at least one embodiment herein;

FIG. 1C depicts an end view of the sleeve of FIG. 1A, as described in at least one embodiment herein

FIG. 1D depicts a side view of one possible configuration of the sleeve positioned on the endoscope or the catheter of FIG. 1A, as described in at least one embodiment herein;

FIG. 1E depicts a perspective view of one possible configuration of the sleeve positioned on the endoscope or the catheter of FIG. 1A, as described in at least one embodiment herein;

FIG. 2 depicts a side view of an assembly including a sleeve as described in at least one embodiment herein;

FIG. 3 depicts a side view of an assembly including a sleeve as described in at least one embodiment herein;

FIG. 4 depicts a side view of an assembly including a sleeve as described in at least one embodiment herein;

FIG. 5 depicts a side view of an assembly including a sleeve as described in at least one embodiment herein; and

FIG. 6 depicts a side view of an assembly including a sleeve as described in at least one embodiment herein.

### DETAILED DESCRIPTION

The present disclosure describes a sleeve configured to be used with either an endoscope or a catheter. The sleeve is designed to aid in aligning a surgical instrument with a target tissue via an endoscope or catheter. The endoscope as described herein may be a bronchoscope.

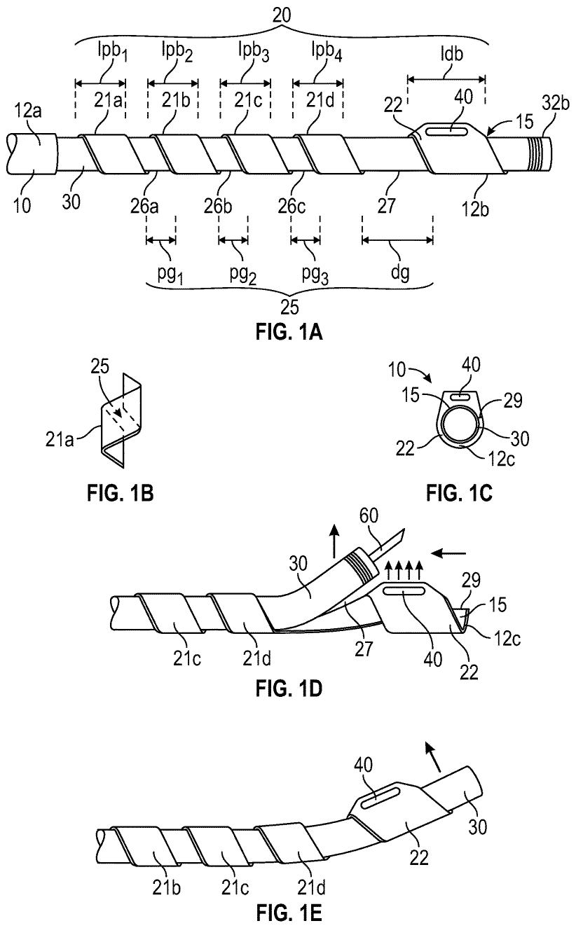

FIGS. 1A-1E depict a sleeve 10 configured for use with an endoscope or catheter 30 as described herein. The sleeve 10 includes a sleeve body 12 with a proximal end portion 12a and a distal end portion 12b. Both the proximal end portion 12a and the distal end portion 12b of the sleeve 10 are configured to be positioned on a portion of an endoscope or catheter 30 with at least the distal end portion 32b of the endoscope or catheter 30 extending therethrough.

At least the distal end portion 12b includes a helical sidewall 20 defining a plurality of helical bands 21a-d, 22 which extend both helically and longitudinally to define a sleeve channel 15 therein. The sleeve channel 15 is configured to receive the endoscope or catheter 30 therein and/or therethrough. A helical slot 25 follows the helical sidewall 20 creating space between each of the plurality of helical bands 21a-d, 22. As shown in FIG. 1B, each helical band is configured to independently form a spiral loop configuration with a slot 25 therebetween, the band having a sufficient length to define a workable channel for maintaining at least a portion of an endoscope or catheter therein.

The plurality of helical bands 21a-d, 22 include one or more proximal helical bands 21a-d and a distal-most helical band 22. Any number of proximal helical bands is envisioned. In some embodiments, the distal-most helical band 22 defines a length ldb greater than a length l_{pb1-4} of the one or more proximal helical bands 21a-d. In some embodiments, the length l_{pb1-4} of each of the one or more proximal helical bands is the same.

As further depicted in FIG. 1A, the helical slot 25 includes a plurality of helical gaps 26a-c, 27 separating each of the neighboring helical bands 21a-d, 22 longitudinally and/or helically. The proximal helical bands 21a-d are separated from each other by a proximal helical gap 26a-c having a gap length pg₁₋₃. The distal-most helical band 22 is separated from the distal-most proximal helical band 21d by a distal helical gap 27 having a distal gap length dg. The distal gap length dg of the distal gap 27 is greater than the gap length pg₁₋₃ of any or all of the proximal helical gaps 26a-c. In some embodiments, the proximal gap lengths pgi-3 of the proximal helical gaps 26a-c can be the same.

As can be seen in FIGS. 1C and 1D, the distal end portion 12b of the sleeve 10, and particularly the distal-most helical band 22, ends at distal edge 29 rendering the distal most portion of the sleeve channel 15 open on both the distal end 12c of the sleeve 10, as well as along the distal edge 29.

In FIG. 1D, the endoscope or catheter 30 can be withdrawn proximally (see left pointing arrow) from the sleeve channel 15 of the distal-most helical band 22 and articulated (see angled arrow) to extend through the distal helical gap 27. The distal helical gap 27 is configured to allow for a distal portion of the endoscope or catheter including a surgical instrument 60 to extend therethrough. The endoscope or catheter 30 may possess the ability to articulate and/or form a curve. Such endoscope or catheter are known to those of ordinary skill and may include steerable devices, concentric tube devices, and/or pre-curved devices.

Typically, the distal helical gap 27 includes a distal gap length dg that is at least greater than or equal to a diameter of the endoscope or catheter 30 used therewith. The use of the term diameter is not intended to limit the endoscope or catheter 30 to only a circular shape. Rather, the term diameter is intended to represent the widest or thickest part of the endoscope or catheter generally transverse the longitudinal axis.

In some embodiments, the distal gap length dg of the distal helical gap 27 may be from 1.1 to 3 times greater than the diameter of the endoscope or catheter 30. In some embodiments, the distal gap length dg of the distal helical gap 27 may be from 1.5 to 2.5 times greater than the diameter of the endoscope or catheter 30.

In some embodiments, the distal gap length dg of the distal helical gap 27 may range from about 1 mm to 6 mm. In some embodiments, the distal gap length dg of the distal helical gap 27 may range from about 1.5 mm to 5 mm. In some embodiments, the distal gap length dg of the distal helical gap 27 may range from about 2 mm to 4 mm.

FIG. 1D also illustrates at least one benefit of the various sleeves described herein, which includes the ability to treat and/or biopsy the tissue under direct ultrasound. As specifically shown in FIG. 1D, the sleeve 10, and particularly the distal-most helical band 22, may include one or more US transducers 40. The US transducer is configured to be positioned directly beneath the endoscope or catheter 30 (including a surgical instrument 50) when extending from the distal helical gap 27 into the tissue surrounding the distal end portion 12b of the sleeve 10.

In some embodiments, the endoscope or catheter 30 can be articulated or steered into a curved configuration free of the sleeve 10 so the sleeve 10 does not articulate or curve with the endoscope or catheter 30 (FIG. 1D). In some embodiments, the endoscope or catheter 30 can be maintained within the sleeve channel 15 of the distal-most helical band 22 while the endoscope or catheter 30 is articulated or steered into a curved configuration. In such embodiments, the sleeve 10 may curve or bend with the endoscope or catheter 30 (FIG. 1E).

In some embodiments, the proximal end portion 12a of the sleeve body 12 is a tubular portion free of any helical bands. In some embodiments, the one or more proximal helical bands 21a-d additionally form the proximal end portion 12a of the sleeve body 12 such that the entire sleeve body 12 includes only a plurality of helical bands.

In some embodiments, the longitudinal sleeve channel may narrow from the proximal end portion to the distal end portion of the sleeve. In some embodiments, the longitudinal sleeve channel may remain of constant size from the proximal end portion to the distal end portion of the sleeve.

The sleeves described herein may be made of any biocompatible material. In some embodiments, the sleeves may be made from a hard plastic. In some embodiments, the sleeves may be made from a polymeric material including, but not intended to be limited to, polyolefins (such as polypropylene), polystyrene, polyvinyl chloride, polyamides (such as polyether block amides), polyurethane, polycarbonate, polyethylene terephthalate and combinations thereof. polyolefins such as polypropylene. low density polypropylene, high density polypropylene can be used. Alternatively, vinyl polymers such as polymethyl methacrylate, polystyrene, polyvinyl chloride can be used. As another alternative, polyamides such as polyether block amides can be used. As another alternative, polyesters such as polyurethane, polycarbonate, polyethylene terephthalate glycol, polybutylene terephthalate, polyethylene terephthalate are used. The materials may include a hardness sufficient to allow passage of the helical sleeves through tissue without misalignment of the sleeve, i.e., such as folding or rolling of the sleeve over itself. In some embodiments, the

In addition, the helical design of the sleeve provides a flexibility not found in solid or non-helical sleeves of a similar hardness so that the sleeves herein can be used with curved and/or steerable catheters. The various helical sleeves described herein may be articulated or steered by the catheter positioned with the sleeve channel and/or may be articulated or steered, independent of the catheter/surgical instrument, by the incorporation of one or more guidewires thereto. In some embodiments, the one or more guidewires may be incorporated with the distal- most helical band.

The sleeves may also be formed using any suitable method, including but not limited to, extrusion, pressing, molding, casting, and the like. In some embodiments, the sleeves may be formed by a molding process.

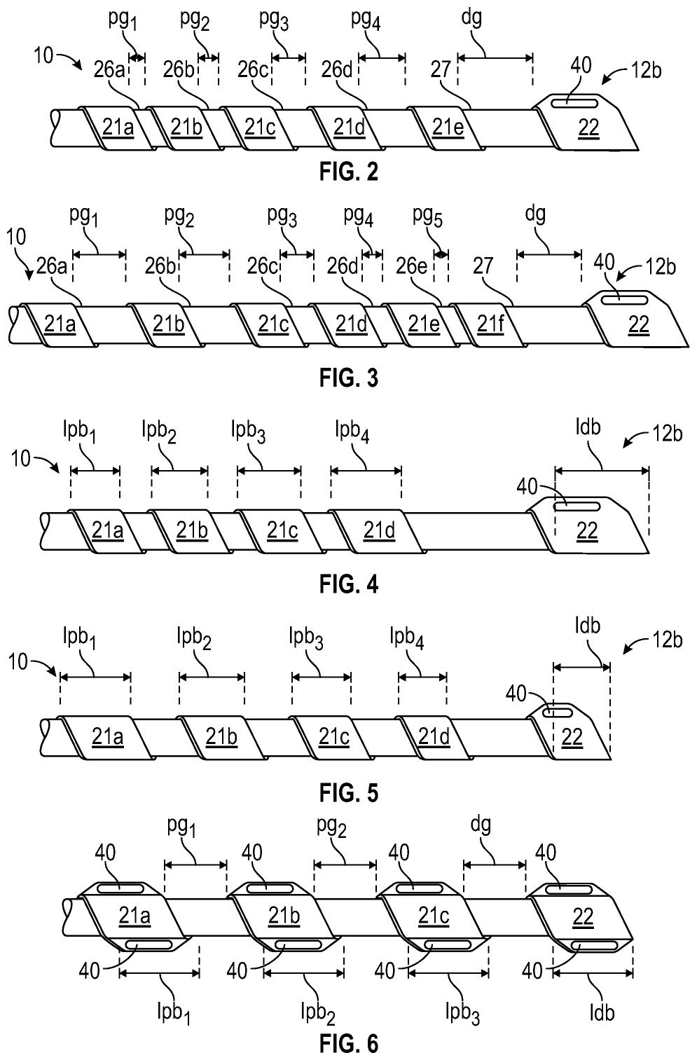

Turning now to FIGS. 2-6, variations of the sleeves 10 described herein are depicted. Generally, FIGS. 2-6 will be described with reference to those portions which differ from the sleeve 10 of FIGS. 1A-1D. For example, in some embodiments, as illustrated in FIG. 2, the distal end portion 12b includes a plurality of proximal helical bands 21a-e separated longitudinally by proximal helical gaps 26a-d, wherein the length pg₁₋₄ between each of the one or more proximal helical bands 26a-e increases as the gaps move distally along the sleeve, i.e., pg₁<pg₂<pg₃<pg₄. In some embodiments, pg₁<pg₂<pg₃<pg₄<dg. In such embodiments, the sleeve may be stiffer near the proximal end portion 12a than the distal end portion 12b increasing the flexibility of the distal end portion 12b.

In FIG. 3, in some embodiments, the distal end portion 12b includes a plurality of proximal helical bands 21a-f separated longitudinally by proximal helical gaps 26a-f, wherein the length pg₁₋₅ between each of the one or more proximal helical bands 26a-f decreases as the gaps move distally along the sleeve, i.e., pg₁>pg₂>pg₃>pg₄>pg₅. In some embodiments, dg>pg₁>pg₂>pg₃>pg₄>pg₅. In such embodiments, the sleeve may be stiffer near the distal end portion 12b than the proximal end portion 12a decreasing the flexibility of the distal end portion 12b.

In FIG. 4, in some embodiments, the distal end portion 12b includes a plurality of proximal helical bands 21a-d and a distal-most helical band 22, a length l_{pb1-4} of each of the individual helical bands 21a-d increases as the bands move distally along the sleeve, i.e., l_{pb1}<l_{pb2}<l_{pb3}<l_{pb4}. In some embodiments, l_{pb1}<l_{pb2}<l_{pb3}<l_{pb4}<l_{db}. In such embodiments, the sleeve may be stiffer near the distal end portion 12b than the proximal end portion 12a decreasing the flexibility of the distal end portion 12b.

In FIG. 5, in some embodiments, the distal end portion 12b includes a plurality of proximal helical bands 21a-d and a distal-most helical band 22, a length l_{pb1-4} of each of the individual helical bands 21a-d decreases as the bands move distally along the sleeve, i.e., l_{pb1}>l_{pb2}>l_{pb3}>l_{pb4}. In some embodiments, l_{pb1}>l_{pb2}>l_{pb3}>l_{pb4}. In such embodiments, the sleeve may be stiffer near the proximal end portion 12a than the distal end portion 12b increasing the flexibility of the distal end portion 12b.

In FIG. 6, in some embodiments, the proximal helical bands 21a-21c and the distal-most helical band 22 define a common length, i.e., l_{pb1}=l_{pb}=l_{pb3}=l_{db} and/or the proximal helical bands 21a-21c and the distal-most helical band 22 are separated by proximal helical gaps 26a-c and distal helical gap 27 having a common length, i.e., pg₁=pg₂=pdg. In such embodiments, unlike some of the other embodiments depicted herein, the length of the proximal helical gaps and distal helical gap are sufficient to allow for the passage of the catheter or surgical instrument to extend therethrough along any portion of the sleeve (as opposed to only the distal helical gap only) and still be monitored by the one or more US transducers 40.

The one or more US transducers are configured to transmit ultrasound waves and/or receive reflected ultrasound waves. Generally, the ultrasound waves penetrate the tissue surrounding the distal end portion of the sleeve based on the frequency of the ultrasound waves. For example, 1 megahertz (MHz) ultrasound waves penetrate to a depth of 2 cm to 5 cm and 3 MHz ultrasound waves penetrate to a depth of 1.5 cm.

Generally, the US waves are reflected at a boundary where density changes or at the interface between tissues. The US transducers are intended to be used at least during treatment and/or biopsy. In additional, the US transducers may be used to assist with navigating the sleeve to a particular tissue. When navigating luminal tissue, such as the lungs, the US waves are reflected from the inside wall of a bronchial tree, from the outside wall of the bronchial tree, and from a diseased portion or cancerous portion located at the outside wall of the bronchial tree and provide finite details of the lung structure and the tissue patency that could not otherwise be revealed using non-invasive imaging means. When treating or biopsying a target tissue, the US transducers and the waves emitted therefrom can be used identify at least one of the distal end portion of the endoscope or catheter extending through the distal helical gap of the sleeves described herein or the tissue surrounding the distal end portion of the sleeve and particularly near the distal-most helical band and/or the distal helical gap. Any suitable wired or wireless US transducer may be used. Some non-limiting examples include a radial transducer, a linear transducer, a piezoelectric transducer, and the like.

In some embodiments, the sleeves described herein may further include a flexible printed circuit board (PCB) for making electrical connection with and/or driving the US transducers. It is envisioned that since the PCB is flexible, the flexible PCB is configured to extend along a length of the sleeve body, while maintaining the ability to change configurations, like the sleeve body. In some embodiments, the flexible PCB is located within and/or extends along the body of the helical bands described herein. The inclusion of a flexible PCB does not hinder the sleeves adaptability to accommodate endoscopes or catheters of varying sizes and/or dimensions.

The sleeves described herein are configured to help guide the endoscopes and/or catheters to a proper alignment to a target tissue. The endoscopes or catheters may include one or more surgical instruments configured to locate, biopsy, or treat the target tissue. For example, the surgical instrument may be selected from the group consisting of a locating guide, an imaging device, a guidewire, a surgical balloon, a biopsy forceps, a cytology brush, an aspirating needle, an ablation device, and combinations thereof.

The sleeves described herein are configured to be combined with a variety of endoscope or catheters to form a sleeved endoscope assembly or a sleeved catheter assembly. Each of the assemblies further optionally including one or more surgical instruments. The sleeved endoscope assemblies and/or sleeved catheter assemblies are suitable for use with electromagnetic navigation systems for visualizing a luminal network of a patient, and/or particularly a lung of a patient. The addition of the sleeves described herein provides better alignment and additional clarity with respect to the target tissue adjacent the sleeve which can result in different treatment options being considered to avoid adversely affecting the adjacent tissue.

The sleeves described herein may also be included in a kit. In some embodiments, a kit may include at least one sleeve and an endoscope, such as a bronchoscope, and/or a catheter. The kit may further optionally include one or more surgical instruments.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting but merely as exemplifications of particular embodiments. Those skilled in the art will envision other modifications within the scope and spirit of the claims appended hereto.

What is claimed is:

1. A sleeve configured for positioning on an endoscope comprising:
a sleeve body including a proximal end portion and a distal end portion, at least the distal end portion including a helical sidewall defining a plurality of helical bands extending longitudinally to define a longitudinal sleeve channel therein, the longitudinal sleeve channel configured to receive a portion of the endoscope therein.

2. The sleeve of claim 1, further comprising a helical slot following the helical sidewall separating each of the plurality of helical bands from each other.

3. The sleeve of claim 2, wherein the helical slot comprises a plurality of helical gaps, each gap separating neighboring helical bands of the plurality of helical bands.

4. The sleeve of claim 3, wherein the plurality of helical bands are separated by the plurality of helical gaps having a common length.

5. The sleeve of claim 3, wherein the plurality of helical bands includes one or more proximal helical bands and a distal-most helical band, wherein the distal-most helical band defines a length greater than each of the one or more proximal helical bands.

6. The sleeve of claim 5, wherein a length of each of the one or more proximal helical bands is the same.

7. The sleeve of claim 5, wherein a length of each of the one or more proximal helical bands increases as the proximal helical bands move distally along the sleeve.

8. The sleeve of claim 5, wherein a length of each of the one or more proximal helical bands decreases as the proximal helical bands move distally along the sleeve.

9. The sleeve of claim 5, wherein the plurality of helical gaps includes one or more proximal helical gaps and a distal helical gap, wherein the one or more proximal helical gaps is between neighboring proximal helical bands and the distal helical gap is between the distal-most proximal helical band and the distal-most helical band.

10. The sleeve of claim 9, wherein a proximal gap length of each of the proximal helical gaps is the same.

11. The sleeve of claim 9, wherein a proximal gap length of each of the proximal helical gaps increases as the proximal helical gaps move distally along the sleeve.

12. The sleeve of claim 9, wherein a proximal gap length of each of the proximal helical gaps decreases as the proximal helical gaps move distally along the sleeve.

13. The sleeve of claim 9, wherein a distal gap length of the distal helical gap is greater than a proximal gap length of the proximal helical gaps.

14. The sleeve of claim 5, wherein the distal-most helical band further comprises one or more US transducers.

15. The sleeve of claim 5, wherein a distal-most portion of the longitudinal sleeve channel is open both longitudinally and along a distal edge of the sleeve body.

16. The sleeve of claim 5, wherein the proximal end portion of the sleeve body is configured to receive at least a portion of an endoscope therein.

17. The sleeve of claim 15, wherein the one or more proximal helical bands further extend across the proximal end portion of the sleeve body such that the entire sleeve body includes a plurality of helical bands.

18. The sleeve of claim 1, wherein the longitudinal sleeve channel narrows from the proximal end portion to the distal end portion of the sleeve body.

19. The sleeve of claim 1, wherein the longitudinal sleeve remains of constant size from the proximal end portion to the distal end portion of the sleeve body.

20. A sleeve configured for positioning on a catheter comprising:
a sleeve body including a proximal end portion and a distal end portion, at least the distal end portion including a helical sidewall defining a plurality of helical bands extending longitudinally to define a longitudinal sleeve channel therein, the longitudinal sleeve channel configured to receive a portion of the catheter therein.

21. A kit comprising:
a sleeve including a sleeve body having a proximal end portion and a distal end portion, at least the distal end portion including a helical sidewall defining a plurality of helical bands extending longitudinally to define a longitudinal sleeve channel therein, the longitudinal sleeve channel configured to receive a portion of an endoscope or catheter therein;
at least one of an endoscope or a catheter; and
optionally a surgical instrument.
